SEQUENCE LISTING (2 sequences)

SEQ ID NO: 1 - protein, 5 aa, Artificial Sequence
  Synthetic peptide
DFKRG

SEQ ID NO: 2 - protein, 9 aa, Artificial Sequence
  Synthetic peptide
CGPLGLLAR

(19) United States
(12) Patent Application Publication
(10) Pub. No.: US 2007/0212305 A1
(43) Pub. Date: Sep. 13, 2007

(54) Optical Imaging Contrast Agents For Imaging Lung Cancer

(75) Inventors: Jo Klaveness, Oslo (NO); Edvin Johannesen, Oslo (NO); Helge Tolleshaug, Oslo (NO)

Correspondence Address: GE HEALTHCARE, INC., IP DEPARTMENT, 101 CARNEGIE CENTER, PRINCETON, NJ, 08540-6231, US

(73) Assignee: AMERSHAM HEALTH AS, Oslo (NO)

(21) Appl. No.: 10/582,893

(22) PCT Filed: Dec. 17, 2004
(86) PCT No.: PCT/NO04/00392

§ 371 Date: Dec. 7, 2006

(30) Foreign Application Priority Data
Dec. 18, 2003 (NO) 20035681

(51) Int. Cl. A61K 49/04

(52) U.S. Cl. 424/9.341

(57) ABSTRACT

The invention provides contrast agents for optical imaging of lung cancer in patients. The contrast agents may be used in diagnosis of lung cancer, for follow up of progress in disease development, for follow up of treatment of lung cancer and for surgical guidance. Further, the invention provides methods for optical imaging of lung cancer in patients.

### FIELD OF THE INVENTION

The present invention provides contrast agents for optical imaging of lung cancer in patients. The contrast agents may be used in diagnosis of lung cancer, for follow up of progress in disease development, and for follow up of treatment of lung cancer.

The present invention also provides new methods of optical imaging of lung cancer in patients, for diagnosis and for follow up of disease development and treatment of lung cancer.

### DESCRIPTION OF RELATED ART

Lung cancer is the leading cause of cancer death worldwide. Approximately 25% of all cancer deaths are attributed to lung cancer, and in USA alone, more than 160 000 new cases were diagnosed in year 2000 and more than 150 000 Americans died the same year from lung cancer. Worldwide more than 1 million people died from lung cancer in year 2000.

In general, the prognosis for patients with lung cancer is poor with a 5-year survival rate of less than 15%. Nearly 90% of cases of lung cancer are attributed to cigarette smoking.

Lung cancer can be divided into two distinct forms; small cell lung cancer (SCLC) and non-small cell lung cancer (NSCLC). SCLC is without treatment the most aggressive form of pulmonary tumours with median survival from diagnosis of two to four months. Compared with other forms of lung cancer, SCLC is usually more spread at time of diagnosis but is more responsive to chemotherapy and irradiation. Chemotherapy of SCLC improves the survival time at least four to five fold. At the time of diagnosis about one third of the patients have metastases in other organs. Treatment of SCLC includes radiation therapy and chemotherapy. Typical drugs used in treatment of SCLC include cisplatin, vincristine, doxorubicin, etoposide and cyclophosphamide.

Non-small cell lung cancer (NSCLC) is a common terminology for various classes of lung cancer including epidermoid carcinoma, adenocarcinoma and large cell carcinoma. The disease can be treated in different ways depending on the stage of disease at time of diagnosis. At an early stage the patient can undergo surgery as this group of patients has the best prognosis. At a later stage the patients are usually treated with radiation therapy often in combination with chemotherapy. If the patients have metastases at the time of diagnosis they do not undergo surgery but are treated with radiation therapy or chemotherapy for palliation of symptoms from the primary tumour.

Chemotherapeutic agents used for treatment of NSCLC include paclitaxel, docetaxel, topotecan, irinotecan, vinorelbine and gemcitabine.

Pulmonary function testing including spirometry and DLCO (diffusion capacity of the lung for carbon monoxide) is part of routine evaluation of lung cancer.

Conventional diagnostic staging of suspected lung malignancies involves chest radiography, bronchoscopy, CT of chest, ultrasound bone scans and MRI. MRI is generally more sensitive than CT for diagnosis and staging of lung cancer. Recent advantages in diagnostic imaging of lung cancer include staging of the disease using PET and 18-fluorodeoxyglucose (FDG).

New bronchoscopic techniques like laser-induced fluorescence endoscope (LIFE) bronchoscopy have the potential to improve the diagnosis of lung cancer.

Some methods have been described directed to measurements of lung function using light. U.S. Pat. No. 4,646,750 (Williams) describes a method for detection of pulmonary inflammation using breath luminescence. U.S. Pat. No. 5,227,308 (University of Hawaii) is drawn to a method for assessing lung maturity using fluorescence from naphthalene-based probes. U.S. Pat. No. 5,606,969 (Brigham & Women's Hospital) relates to methods for measuring lung function using diffused light. U.S. Pat. No. 4,534,360 (Williams) relates to a method for detection of lung cancer using breath luminescence.

The following documents describe compounds and methods for diagnosis for lung cancer. U.S. Pat. No. 6,426,072 (Corixa) relates to compositions and methods for the therapy and diagnosis of lung cancer using lung tumour proteins and related substances. The document does not suggest imaging.

U.S. Pat. No. 6,517,811 (Research Corporation Technologies) relates to compounds of cancer imaging and therapy including among others lung cancer. The compounds bind to a cell surface sigma receptor. Compounds including a radionuclide are described.

U.S. Pat. No. 6,509,448 (Corixa) describes compositions and methods for the therapy and diagnosis of lung cancer. The compounds include polypeptides, polynucleotides encoding the polypeptides, antibodies, antigen presenting cells and immune system cells. The patent does not disclose optical imaging contrast agents.

U.S. Pat. No. 6,509,316 (George Washington University) discuss compositions, methods and kits for treatment and diagnosis of lung cancer based on uteroglobin, for preventing/inhibiting metastasis of lung tumor cells. The patent does not describe optical imaging.

R. Baumgartner et al, J. Photochem Photobiol B 1996 36 169-74 studied the effect of inhaled 5-aminolevulinic acid to improve detection of early-stage lung cancer. 5-aminolevulinic acid is not fluorescent, but is a biosynthetic precursor of the fluorescent protoporphyrin IX.

Lung cancer is still a challenge to diagnose and treat. There is a need for improved diagnostic methods, especially for diagnosis of lung cancer in an early stage with good reliability. Surprisingly, we have discovered that the use of optical imaging methods with new contrast agents fulfill these requirements.

### SUMMARY OF THE INVENTION

The present invention provides an optical imaging contrast agent with affinity for an abnormally expressed, biological target associated with lung cancer.

The invention is also described in the claims.

The following definitions will be used throughout the document:

Lung cancer tissue: Includes the two main forms small-cell lung cancer (SCLC) and non small-cell lung cancer (NSCLC), the latter including adenomas and squamous cell carcinomas. It further includes metastases to the lungs from other types of cancer.

Abnormally expressed target: A target that is either overexpressed or downregulated in lung cancer tissue.

Overexpressed target: A receptor, an enzyme or another molecule or chemical entity that is present in a higher amount in lung cancer tissue than in normal tissue.

Downregulated target: A receptor, an enzyme or another molecule or chemical entity that is present in a lower amount in lung cancer tissue than in normal tissue.

### DETAILED DESCRIPTION OF THE INVENTION

A first aspect of the present invention is an optical imaging contrast agent for imaging of lung cancer. By the term optical imaging contrast agent, or just contrast agent, we mean a molecular moiety used for enhancement of image contrast in vivo comprising at least one moiety that interacts with light in the ultraviolet, visible or near-infrared part of the electromagnetic spectrum.

The contrast agent has affinity for an abnormally expressed target associated with lung cancer.

Lung cancer tissue containing a downregulated target is identified by a low amount of bound imaging agent compared to normal tissue. In this situation, the amount of imaging agent should be less than 50% of that in normal tissue, preferably less than 10%.

Preferred contrast agents according to the invention, have affinity for an overexpressed target associated with lung cancer. Preferred targets are those targets that are more than 50% more abundant in lung cancer tissue than in surrounding tissue. More preferred targets are those targets that are more than two times more abundant in lung cancer tissue than in surrounding tissue. The most preferred targets are those targets that are more than 5 times more abundant in lung cancer tissue than in surrounding tissue.

In a further aspect of the invention, targets that are mutated in lung cancer tissue may be identified by lack of binding of an imaging agent that does bind to normal tissue; alternatively, the imaging agent might be directed specifically towards the mutated target, and binding to normal tissue would be minimal. The mutated target can be a protein in lung cancer tissue that is altered as a result of a germline or somatic mutation, and including alterations resulting from differential splicing of RNA and changes in post-translational modifications, particularly glycosylation patterns, but not limited to these types of alterations.

Relevant groups of targets are receptors, enzymes, nucleic acids, proteins, lipids and other macromolecules as, for example, lipoproteins and glycoproteins. The targets may be located in the vascular system, in the extracellular-space, associated with cell membranes or located intracellularly.

Preferred groups of targets are adhesion molecules and extracellular matrix proteins, antigens, proteins involved in cell cycle control and DNA repair, enzymes and inhibitors, hormones and hormone-related proteins, oncogens and receptors associated with lung cancer.

The following biological targets-are overexpressed in lung cancer tissue and are preferred targets for contrast agents for optical imaging of lung cancer:

Adhesion Molecules and Extracellular Matrix Proteins

CD44, CD44v3, CD44v6, ED-B fibronectin, galectin-3, galectin-4, LGALS3 (Galectin) gene, P-selectin, liver-intestinal cadherin 17 and integrins, such as αᵥβ₃. and αᵥβ₅.

Antigens

CA 15.3; CA 72.4, cancer antigen 125 (CA125), CA19-9, carbohydrate antigen 549 (CA 549), carcinoembryonic antigen (CEA), CD105, CD24, CD34, chromogranin A antigens, ki-67, melanoma antigen E tumor-associated antigen, MUC1 (glycosylated mucin), oncoprotein 18, squamous cell carcinoma antigen (SCC), tissue polypeptide antigen (TPA), 5T4 oncofetal trophoblast glycoprotein, insulinoma-associated gene 1 product, FOS-related antigen 1, H/Le^{y}/Le^{b}.

Proteins Involved in Cell Cycle Control and DNA Repair

K-ras, 34cdc2, Bax, bcl-2, Cdc 25A, cdc 25B, Cyclin B1, D1, E, cyclin D, p53, p27, pRb2/p130, retinoblastoma protein, telomerase, thyroid transcription factor 1, CDC6.

Enzymes and Inhibitors

Cyclophilin A, alpha-1 protease inhibitor, arylamine N-acetyltransferase, Bcl2, carbonic anhydrase I and II, carbonic anhydrase-9, caspase-9 and -3, choline kinase, cyclo-oxygenase-2 (COX-2), CYP1A1, CYP2C40, cytidine deaminase, cytochrome P450, deoxycytidine deaminase, dual-specificity yrosine-(Y)-phosphorylation regulated kinase 2 (DYRK 2), glutathione peroxidase, glutathione-S-transferase, GSTP1, GST-pi, helix-loop-helix ubiquitous kinase (CHUK), M2-PK (pyruvate kinase), matrix metalloproteinases (MMPs), MMP-14, collagenase, MMP-9, Stromelysin-3 MutT homologue (hMTH1), an 8oxo-dGTPase, myeloperoxidase, Neuron-specific enolase, phosphatidylinositol-3-kinase, prostaglandin E synthase, spermidine/spermine N1-acetyltransferase (SSAT), superoxide dismutase, thioether S-methyltransferase, tyrosine kinase, urokinase plasminogen activator, ribonucleotide reductase, cystatin C, ERCC1 gene product, dopa decarboxylase, kallikrein 11, ornithine decarboxylase 1, cathepsin H, catepsin L, farnesyl transferase, ribonucleotide reductase, tissue plasminogen activator, glutaminyl cyclase, pronapsin A, carbonyl reductase, leukotriene B4 12 dehydrogenase, thioredoxin reductase, glutathione peroxidase, glycinarnide ribonucleotide formyltransferase (GARFT), thymidylate synthase, dihydrofolate reductase, carboxypeptidase E, proprotein convertase, protein kinase C-alpha, ERCC1 gene product, ERCC2, hMLH1, hMSH2.

Hormones and Hormone-Related Proteins

Arginine vasbpressin, angiopoietin 1, angiopoietin 2, chromogranin A (CgA), CXC chemokines, ghrelin (growth hormone releasing peptide), interferon regulatory factor 1, macrophage migration inhibitory factor, pro-gastrin-releasing peptide (Pro-GRP), RANTES, vascular endothelial growth factor (VEGF), Insulin-like growth factor binding protein 3 (IGFBP3), gastrin-releasing peptide, Cholecystokinin, neurotensin Insulin-like growth factor binding protein 3 (IGFBP3), calcitonin-related polypeptide and somatostatin.

Oncogenes

c-erbB-2, c-kit protein, EphA2 receptor tyrosine kinase, HER2/epidermal growth factor receptor (EGFR), HER-2/neu.

Receptors

Cholecystokinin A receptor, cholecystokinin B receptor, EGFR tyrosine kinase, epidermal growth factor receptor (EGFR), Notch3, TIE-2 precursor, SSR1 signal sequence receptor-α, c-myc protein, Gastrin-releasing peptide receptor, neuromedin B receptor, bombesin receptor, neurotensin receptor, urokinase plasminogen activator receptor, vasopressin receptor, the angiopoietin receptors, vascular endothelial growth receptor (VEGFR), bradykinin receptor.

Other Targets

Achaete scute homolog 1, alpha-1 PI2, alpha-adaptin, aryl hydrocarbon receptor, ataxia-telangectasia D-associated protein, AVP, BAG-1, beta-tubulin III, chromogranin-A, CYFRA, cytochrome b5, cytokeratin 19 fragment (Cyfra21-1), dickkopf homolog 1, differentiated embryo-chondrocyte, expressed gene 1 (DECI) protein, dyskerin, eIF4E (translation initiation factor), epithelial mucin 1, ERK-1, ferritin, GRP, heat-shock proteins, hnRNP A2/B1, heterogeneous nuclear ribonucleoproteins, hnRNP B1, HSP70, HSP90, hypoxia-inducible factor (HIF) 1alpha, JAK-1, L523S.(RNA-binding protein), MDR drug efflux/degradation, metallothionein, napsin A (TA02), NFAT1, p120, P16, proliferating cell nuclear antigen (PCNA), RAD21 homologue, retinoic acid receptor alpha, RhoA, ribonucleoprotein B1, S100 calcium-binding protein P; Solute carrier family 7, member 5, SpA, stanniocalcin 1, stathmin, surfactant proteins A, B, C and D, synaptophysin, thyroid transcription factor-1 (TTF-1), transmembrane protein 63 kD (ER/Golgi), UDG, uroplakin II, AKT, Ras, Ras-association domain family 1 (RASSF1A) protein, AFP, ALG-2, CC10, Kinin B1, MRP4, Nm23H1 gene.

Some targets are downregulated in lung cancer tissue and preferred targets are: Forkhead protein FREAC-1, Cadherin 5, Laminin 131, Placenta copper monoamine oxidase, ABC3 ATP-binding cassette 3, Surfactant protein SP-C1, RAGE.

Among the more preferred targets for contrast agents for optical imaging of lung cancer are: Galectin-3, cancer antigen 125 (CA125), cathepsin L, MUC1, caspase-9 and -3, cyclo-oxygenase-2 (COX-2), glutathione-S-transferase (GST), the angiopoietin receptors, integrin αvβ3, vascular endothelial growth factor receptor (/EGF), HER2/epidermal growth factor receptor (EGFR), MDR, urokinase plasminogen activator receptor and cyclin D1.

The most preferred targets for contrast agents for optical imaging of lung cancer are cathepsin L, caspase-3, HER2/epidermal growth factor receptor (EGFR), urokinase plasminogen activator receptor and integrin αvβ3.

Generally, any targets that-have been identified as possible targets for agents for treatment of lung cancer are potential targets also in optical imaging.

Small cell lung cancer (SCLC) synthesizes, and has receptors for several biologically active peptides that may be usable targets. The same receptors may not be relevant for non-small cell lung cancer (NSCLC).

The preferred contrast agents of the present invention are molecules with relatively low molecular weights. The molecular weight of preferred contrast agents is below 14 000 Daltons, preferably below 10000 Daltons and more preferably below 7000 Daltons.

The contrast agents, according to the present invention, are preferably comprised of a vector that has affinity for an abnormally expressed target in lung cancer tissue, and an optical reporter.

Thus viewed from one aspect the present invention provides a contrast agent of formula I:
V-L-R (I)
wherein V is one or more vector moieties having affinity for one or more abnormally expressed target in lung cancer tissue, L is a linker moiety or a bond and R is one or more reporter moieties detectable in optical imaging.

The vector has the ability to direct the contrast agent to a region of lung cancer. The vector has affinity for the abnormally expressed target and preferably binds to the target. The reporter must be detectable in an optical imaging procedure and the linker must couple vector to reporter, at least until the reporter has been delivered to the region of lung cancer and preferably until the imaging procedure has been completed.

The vector can generally be any type of molecules that have affinity for the abnormally expressed target. The molecules should be physiologically acceptable and should preferably have an acceptable degree of stability. The vector is preferably selected from the following group of compounds, peptides, peptoids/peptidomimetics, oligonucleotides, oligosaccharides, lipid-related compounds like fatty acids, traditional organic drug-like small molecules, synthetic or semi-synthetic, and derivatives and mimetics thereof. When the target is an enzyme the vector may comprise an inhibitor of the enzyme or an enzyme substrate. The vector of the contrast agent preferably has a molecular weight of less than 10 000 Daltons, more preferably less than 4500 Daltons and most preferably less than 2500 Daltons, and hence does not include antibodies or internal image antibodies. In addition to problems with immune reactions, long circulation time and limited distribution volume, many antibodies have an affinity for the receptor that is too low for use in imaging.

An optical imaging contrast agent comprising a vector having affinity for any of the preferred targets is a preferred embodiment of the invention.

Contrast agents having affinity for more than one abnormally expressed target related to the disease is an aspect of the invention. Such contrast agents can comprise two or more different vectors or molecular subunits that target two or more different abnormally expressed targets.

Another possibility according to the present invention is that the contrast agent comprises one vector that is able to bind to more than one abnormally expressed target in lung cancer.

A contrast agent according to the present invention can also comprise more than one vector of same chemical composition that bind to the abnormally expressed biological target.

Some receptors are unique to endothelial cells and surrounding tissues. Examples of such receptors include growth factor receptors such as VEGF and adhesion receptors such as the integrin family of receptors. Peptides comprising the sequence arginine-glycine-aspartic acid (RGD) are known to bind to a range of integrin receptors. Such RGD-type peptides constitute one group of vectors for targets associated with lung cancer.

Below are some examples of vectors-having affinity for lung cancer-related abnormally expressed targets:

Vectors for Cyclo-Oxygenase-2 (COX-2): Arachidonic Acid

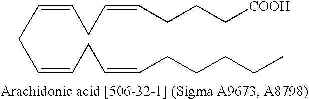

Arachidonic acid is the endogenous substrate for COX-2, and is an essential fatty acid and a precursor in the biosynthesis of prostaglandins.

Other vectors for COX-2 are exogenous compounds that bind to COX-2, for example so-called COX-2 inhibitors. The chemical classes of the main COX-2 inhibitors are shown in WO 02/07721.

Such vectors include:

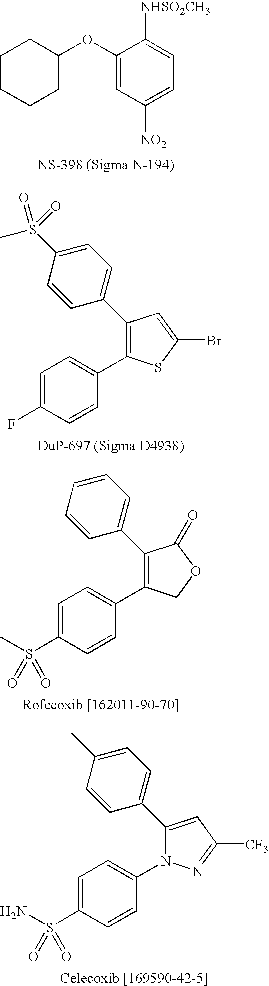

Vectors for Matrix Metalloproteinases, Such as for MMP-7:

Peptide sequence: Cys-Gly-Pro-Leu-Gly-Leu-Leu-Ala-Arg-OH

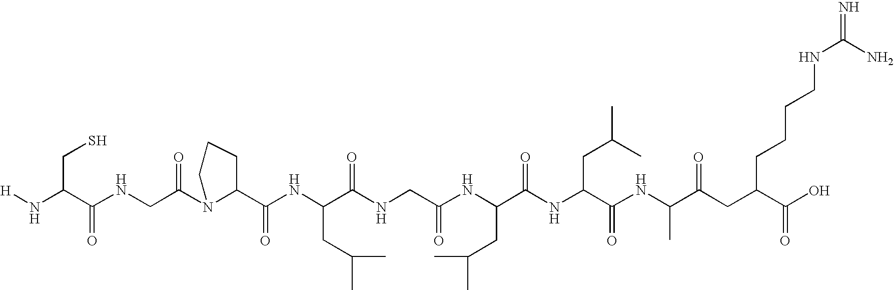

Vectors for Mapping of Tyrosine Kinase Activity of the Epidermal Growth Factor Receptor (EGFR):

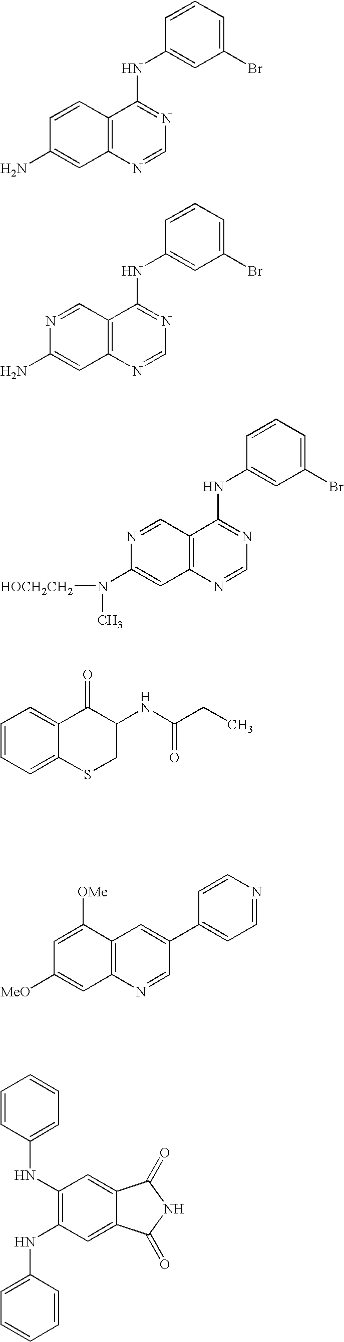

Gefitinib (Iressa®):

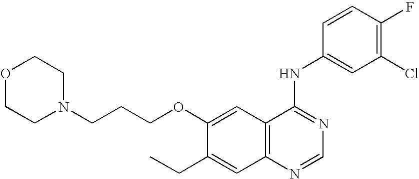

These represent a group of kinase inhibitors and are analogues of ATP.

A wide variety of linkers can be used. The linker component of the contrast agent is at its simplest a bond between the vector and the reporter moieties. In this aspect the reporter part of the molecule is directly bound to the vector that binds to the abnormally expressed target. More generally, however, the linker will provide a mono- or multi-molecular skeleton covalently or non-covalently linking one or more vectors to one or more reporters, e.g. a linear, cyclic, branched or reticulate molecular skeleton, or a molecular aggregate, with in-built or pendant groups which bind covalently or non-covalently, e.g. coordinatively, with the vector and reporter moieties. The linker group can be relatively large in order to build into the contrast agent optimal size or optimal shape or simply to improve the binding characteristics for the contrast agent to the abnormally expressed target in lung cancer tissue.

Thus, linking of a reporter unit to a desired vector may be achieved by covalent or non-covalent means, usually involving interaction with one or more functional groups located on the reporter and/or vector. Examples of chemically reactive functional groups which may be employed for this purpose include amino, hydroxyl, sulfhydroxyl, carboxyl and carbonyl groups, as well as carbohydrate groups, vicinal diols, thioethers, 2-aminoalcohols, 2-aminothiols, guanidinyl, imidazolyl and phenolic groups.

The reporter is any moiety capable of detection either directly or indirectly in an optical imaging procedure. The reporter can be a light scatterer (e.g. a coloured or uncoloured particle), a light absorber or a light emitter. More preferably the reporter is a dye such as a chromophore or a fluorescent compound. The dye part of the contrast agent can be any dye that interacts with light in the electromagnetic spectrum with wavelengths from the ultraviolet light to the near-infrared. Preferably, the contrast agent of the invention has fluorescent properties.

Preferred organic dye reporters include groups having an extensive delocalized electron system, e.g. cyanines, merocyanines, indocyanines, phthalocyanines, naphthalocyanines, triphenylmethines, porphyrins, pyrilium dyes, thiapyrilium dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, iodoaniline dyes, bis(S,O-dithiolene) complexes. Fluorescent proteins, such as green fluorescent protein (GFP) and modifications of GFP that have different absorption/emission properties are also useful. Complexes of certain rare earth metals (e.g., europium, samarium, terbium or dysprosium) are used in certain contexts, as are fluorescent nanocrystals (quantum dots).

Particular examples of chromophores which may be used include fluorescein, sulforhodamine 101 (Texas Red), rhodamine B, rhodamine 6G, rhodamine 19, indocyanine green; Cy2, Cy3, Cy3B, Cy3.5, Cy5, Cy5.5, Cy7, Cy7.5, Marina Blue, Pacific Blue, Oregon Green 488, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750. The cyanine dyes are particularly preferred.

Particularly preferred are dyes which have absorption maxima in the visible or near-infrared region, between 400 nm and 3 μm, particularly between 600 and 1300 nm.

The contrast agents according to the invention can comprise more than one dye molecular sub-unit. These dye sub-units can be similar or different from a chemical point of view. Preferred contrast agents have less than 6 dye molecular sub-units.

Several relevant targets for lung cancer are enzymes. A contrast agent for optical imaging of lung cancer for targeting an enzyme can be an enzyme contrast agent substrate that can be transformed to a contrast agent product possessing different pharmacokinetic and/or pharmacodynamic properties from the contrast agent substrate. This embodiment of the invention provides contrast agent substrates having affinity for an abnormally expressed enzyme, wherein the contrast agent substrate changes pharmacodynamic and/or pharmacokinetic properties upon a chemical modification into a contrast agent product in a specific enzymatic transformation, and thereby enabling detection of areas of disease upon a deviation in the enzyme activity from the normal. Typical differences in pharmacodynamic and/or pharmacokinetic properties can be binding properties to specific tissue, membrane penetration properties, protein binding and solubility properties.

Alternatively, if the abnormally expressed target for diagnosis of lung cancer is an enzyme, the contrast agent for optical imaging can be a dye molecule that directly binds to the enzyme. The contrast agent will have affinity for the abnormally expressed enzyme, and this may be used to identify tissue or cells with increased enzymatic activity.

In a further aspect of the invention, the contrast agent changes dye characteristics as a result of an enzymatic transformation. For example, a fluorescent dye reporter of the contrast agent is quenched (no fluorescence) by associated quencher groups, until an enzymatic cleavage takes place, separating the dye from the quencher groups and resulting in fluorescence at the site of the abnormally expressed enzyme.

Another aspect of this part of the invention is that the dye may change colour, as e.g. a change in absorption and/or emission spectrum, as a result of an enzymatic transformation.

If the abnormally expressed target for diagnosis of lung cancer is a receptor or another non-catalytic target, the contrast agent for optical imaging can bind directly to the target and normally not change the dye characteristics.

The preferred contrast agents of the present invention are soluble in water. This means that the preferred contrast agents have a solubility in water at pH 7.4 of at least 1 mg/ml.

The contrast agents of the present invention can be identified by random screening, for example by testing of affinity for abnormally expressed targets of a library of dye labelled compounds either prepared and tested as single compounds or by preparation and testing of a mixture of compounds (a combinatorial approach). Alternatively, random screening may be used to identify suitable vectors, before labelling with a reporter.

The contrast agents of the present invention can also be identified by use of technology within the field of intelligent drug design. One way to perform this is to use computer-based techniques (molecular modeling or other forms of computer-aided drug design) or use of knowledge about natural and exogenous ligands (vectors) for the abnormally expressed targets. The sources for exogenous ligands can for example be the chemical structures of therapeutic molecules for targeting the same target. One typical approach here will be to bind the dye chemical sub-unit (reporter) to the targeting vector so that the binding-properties of the vector are not reduced. This can be performed by linking the dye at the far end away from the pharmacophore centre (the active targeting part of the molecule).

The contrast agents of the invention are preferably not endogenous substances alone. Some endogenous substances, for instance estrogen, have certain fluorescent properties in themselves, but they are not likely to be sufficient for use in optical imaging. Endogenous substances combined with an optical reporter however, fall within the contrast agents of the invention.

The contrast agent of the invention are intended for use in optical imaging. Any method that forms an image for diagnosis of disease, follow up of disease development or for follow up of disease treatment based on interaction with light in the electromagnetic spectrum from ultraviolet to near-infrared radiation falls within the term optical imaging. Optical imaging further includes all methods from direct visualization without use of any device and use of devices such as various scopes, catheters and optical imaging equipment, for example computer based hardware for tomographic presentations. The contrast agents will be useful with optical imaging modalities and measurement techniques including, but not limited to: luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; interferometry; coherence interferometry; diffuse optical tomography and fluorescence mediated diffuse optical tomography (continuous wave, time domain and frequency domain systems), and measurement of light scattering, absorption, polarisation, luminescence, fluorescence lifetime, quantum yield, and quenching.

Examples of contrast agents for optical imaging of lung cancer according to the invention, and potential synthesis of some of these, are shown below:

Contrast Agents with Affinity for Mapping of COX-2:

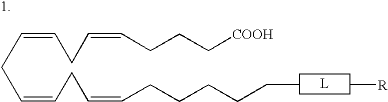

Wherein arachidonic acid, the endogenous substrate for COX-2, is linked to a reporter (R) via a linker (L).

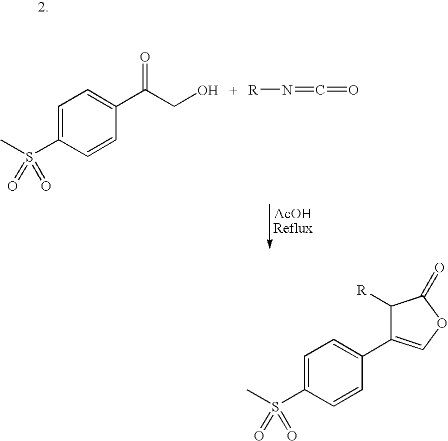

Wherein a COX-2 inhibitor derivative is linked to a reporter. R is any reporter according to the present invention; for example fluorescein, and L is a linker. For this example, giving a Rofecoxib-derivative, a possible synthesis is given.

Contrast Agent for Mapping of Matrix Metalloproteinase

The peptide vector (Cys-Gly-Pro-Leu-Gly-Leu-Leu-Ala-Arg) is linked to e.g. fluorescein (R) through a linker (L):

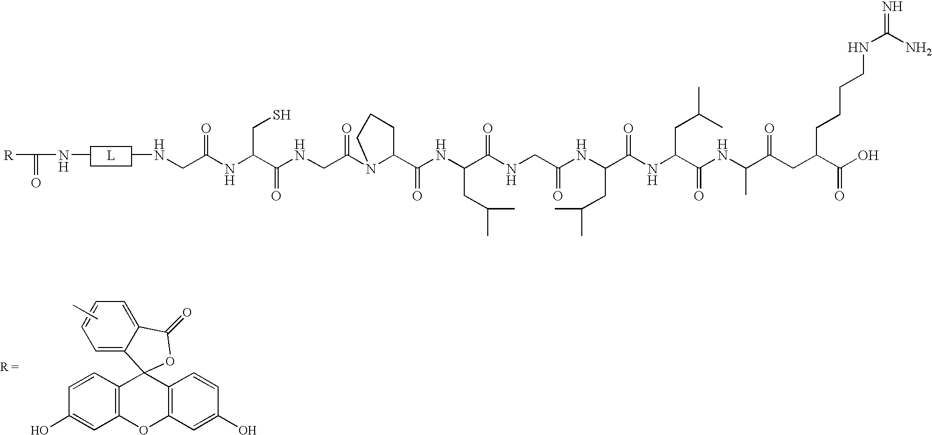

A synthesis is given in example 2.

Contrast Agents for Mapping of Tyrosine Kinase Activity of the Epidermal Growth Factor Receptor (EGFR):

A suggested synthesis is given for preparation of a contrast agent comprising a vector with affinity for tyrosine kinase of the epidermal growth factor linked to a Cy5.5 reporter.

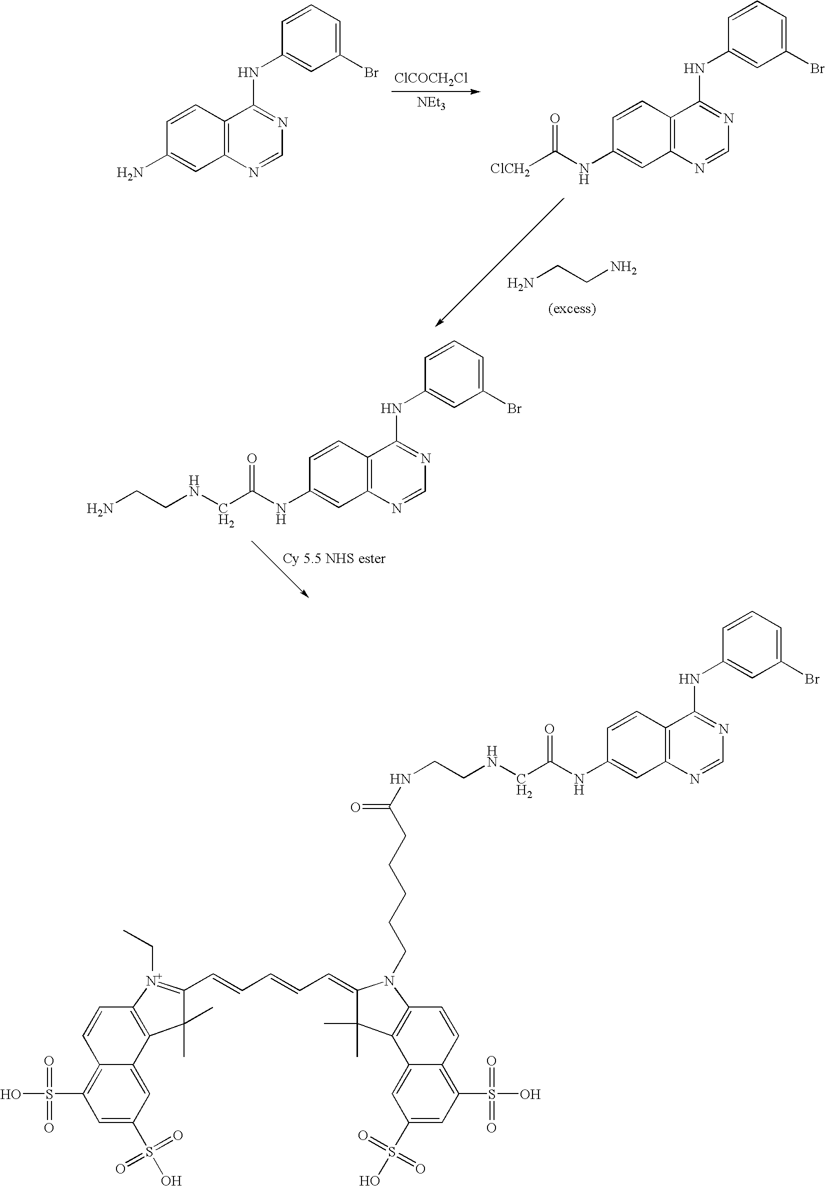

A further embodiment is the use of contrast agents of the invention for optical imaging of lung cancer, that is for diagnosis of lung cancer, for use in follow up the progress in lung cancer development, for follow up the treatment of lung cancer, or for surgical guidance.

In the context of this invention, diagnosis includes screening of selected populations, early detection, biopsy guidance, characterisation, staging and grading. Follow up of treatment includes therapy efficacy monitoring and long-term follow-up of relapse. Surgical guidance includes tumour margin identification during resection.

Still another embodiment of the invention is a method of optical imaging of lung cancer using the contrast agents as described.

Still another embodiment of the invention is a method of optical imaging for diagnosis, to follow up the progress of lung cancer development and to follow up the treatment of lung cancer, using a contrast agent as described.

One aspect of these methods is to administer the present contrast agents and follow the accumulation and elimination directly visually during surgery. Another aspect of these methods is to administer the present contrast agents and perform visual diagnosis through a bronchoscope.

Still another aspect of the present invention is to administer the present contrast agents and perform the image diagnosis using computerized equipment as for example a tomograph.

Still another embodiment of the invention is use of a contrast agent as described for the manufacture of a diagnostic agent for use in a method of optical imaging of lung cancer involving administration of said diagnostic agent to an animate subject and generation of an image of at least part of said body, preferably the lungs or part of the lungs

Still another embodiment of the invention is pharmaceutical compositions comprising one or more contrast agents as described or pharmaceutically acceptable salts thereof for optical imaging for diagnosis of lung cancer, for follow up progress of lung cancer development or for follow up the treatment of lung cancer. The contrast agent of the present invention can be formulated in conventional pharmaceutical or veterinary parenteral administration forms, e.g. suspensions, dispersions, etc., for example in an aqueous vehicle such as water for injections. The agent may also be formulated as an aerosol. Such compositions may further contain pharmaceutically acceptable diluents and excipients and formulation aids, for example stabilizers, antioxidants, osmolality adjusting agents, buffers, pH adjusting agents, etc. The most preferred formulation is a sterile solution for intravascular administration or for direct injection into area of interest. Where the agent is formulated in a ready-to-use form for parenteral administration, the carrier medium is preferably isotonic or somewhat hypertonic.

The dosage of the contrast agents of the invention will depend upon the clinical indication, choice of contrast agent and method of administration. In general, however dosages will be between 1 micro gram and 70 grams and more preferably between 10 micro grams and 5 grams for an adult human.

While the present invention is particularly suitable for methods involving parenteral administration of the contrast agent, e.g. into the vasculature or directly into an organ or muscle tissue, intravenous administration being especially preferred, it is also applicable where administration is not via a parenteral route, e.g. where administration is transdermal, nasal, sub-lingual or is into an externally voiding body cavity, e.g. through the bronchi. The agent may be formulated as an aerosol for administration by inhalation, or may be sprayed on directly during endoscopy. The present invention is deemed to extend to cover such administration.

The following examples are illustrative only and not intended to be limiting. Other features and advantages of the invention will be apparent from the detailed description and from the claims.

### EXAMPLES

### Example 1

### Contrast Agent for Mapping of COX-2 Activity. Synthesis of COX-2 Ligand Coupled to Fluorescein

Step 1

2-Hydroxy-1-(4-methanesulfonylphenyl)ethanone is prepared from 2-bromo1-(4-methanosulfonylphenyl)ethanone according to C. Puig et al in J. Med. Chem 2000, 43 214-223.

Step 2

A solution of 2-hydroxy-1-(4-methanosulfonylphenyl) ethanone (1.50 g, 7 mmol) and fluorescein isocyanate isomer 1 (2.72 g, 7 mmol) is heated in DMF at 120° C. for 5 hours.

The mixture is cooled, DMF evaporated off and acetic acid (25 ml) is added. The mixture is refluxed for 10 hours. The acetic acid is evaporated and the resulting mixture is purified on silica using chloroform/methanol as eluent.

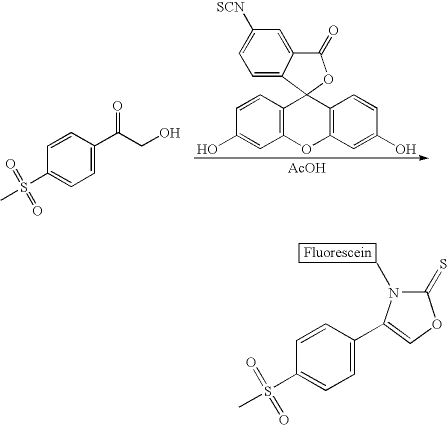

### Example 2

### Contrast Agent for Mapping of Matrix Metalloproteinase (MMP). Synthesis of Fluorescein-Cys-Gly-Pro-Leu-Gly-Leu-Leu-Ala-Arg-OH Linker Conjugate

Step 1

The peptide component was synthesized on an ABI 433A automatic peptide synthesizer starting with Fmoc-Arg(Pmc)-wang resin on a 0.1. mmol scale using 1 mmol amino acid cartridges. The amino acids were pre-activated using HBTU before coupling. An aliquot of the peptide resin was then transferred to a clean round bottom flask an N-methyl morpholine (1 mmol) in DMF (5 ml) added followed by chloroacetyl chloride (1 mmol). The mixture was gently-shaken until Kaiser test negative. The resin was extensively washed with DMF.

Step 2

5(6)carboxyfluorescein (188 mg, 0.5 mmol) and dicyclohexylcarbodiimide (113 mg, 0.55 mmol) are dissolved in DMF (20 ml). The mixture is stirred for 2 hours and cooled to 0° C. A solution of hexamethylenediamide (116 mg, 1 mmol)-and DMAP (30 mg) in DMF is added and the mixture is stirred at ambient temperature for 72 hours. The solution is evaporated and the conjugate between carboxyfluorescein and hexamethylene-amine is isolated as monoamide by chromatography (silica, chloroform and methanol).

Step 3

The resin from step 1 is suspended in DMF (5 ml) and amide-amine conjugate from step 2 (0.5 mmol) pre-dissolved in DMF (5 ml) containing triethylamine (0.5 mmol) is added. The mixture is heated to 50° C. for 16 hours then excess reagents filtered off, following extensive washing with DMF, DCM and diethyl ether then air drying. The product is treated with TFA containing TIS (5%), H₂O (5%), and phenol (2.5%) for 2 hours.

Excess TFA is removed in vacuo and the pepbde is precipitated by the addition of diethyl ether. The crude peptide conjugate is purified by preparative HPLC C C-18, acetonitril, TFA, water).

### Example 3

### Contrast Agent for Binding to p53 Oncoprotein

Step 1. Synthesis of 2,2-bis(hydroxymethyl)-1-aza-bicyclo[2,2,2,]octan-3-one. 3-quinuclidinone hydrochloride (Aldrich Q 190-5) (1 mmol) is dissolved in methanol-water (1:1, 30 ml). An aqueous solution of formaldehyde (37%, 2.5 mmol) and sodium hydroxide (1.5 mmol) are added. The mixture is stirred for 12 hours at 50° C. The solvents are evaporated and the title compound isolated as free base using flash chromatography (silica, ethylacetate/chloroform, hexane).

Step 2.

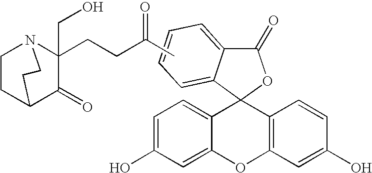

5(6)-carboxyfluorescein (0.1 mmol) and dicyclohexyl carbodiimide (0.11 mmol) are dissolved in DMF. The mixture is stirred for 3 hours and cooled to 0° C. A solution of 2,2-bis(hydrozymethyl)-1-azabicyclo[2,2,2]octane-3-one (0.5 mmol) and DMAP (10 mg) in DMF is added and the mixture is stirred at ambient temperature for 72 hours. The solution is evaporated and the contrast agent is isolate by flash chromatography (silica, ethyl acetate/hexane).

### Example 4

### Contrast Agent for Mapping of Tyrosine Kinase Activity of the Epidermal Growth Factor

Step 1. 4-[(3-bromophenyl)amino]-7-[N-(2-hydroxy-ethyl)-N-methylamino]pyrido [4,3-d]pyrimidine is prepared according to A. M. Thomson et al in J. Med. Chem. (1997) 40 3915-3925.

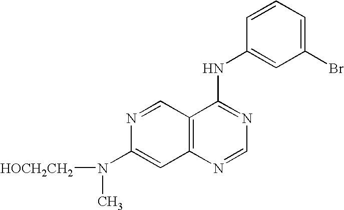

Step 2. 5(6)-carboxyfluorescein (1 mmol), dicyclohexylcarbodiimide (1.2 mmol) and DMAP (50 mg) are dissolved in DMF (30 ml). The mixture is stirred for 24 hours. A solution of the alcohol from step 1 (1 mmol) in DMF (5 ml) is added and the mixture is stirred for 3 days at ambient temperature. The fluorescein ester conjugate with the alcohol vector is isolated by chromatography (silica, hexane/chloroform).

### Example 5

### Contrast Agent for Mapping of EGFR/erB2 Tyrosine Kinase

Step 1. N-[4((3-bromophenyl)amino)quinazolin-7-y-]acrylamide is prepared according to J. B. Smaill et al J. Med. Chem. (1999) 42 1803-1815.

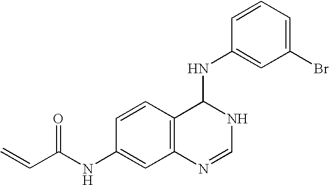

Step 2. N-[4((3-bromophenyl).amino)quinazolin-7-y-]acrylamide from step 1 (1 mmol) and ethylenediamine (10 mmol) are dissolved in DMF (25 ml). The mixture is stirred at 50° C. for 12 hours. The solvent is evaporated off and the conjugate compound is isoloated by flash chromatography (silica, hexane, chloroform, methanol).

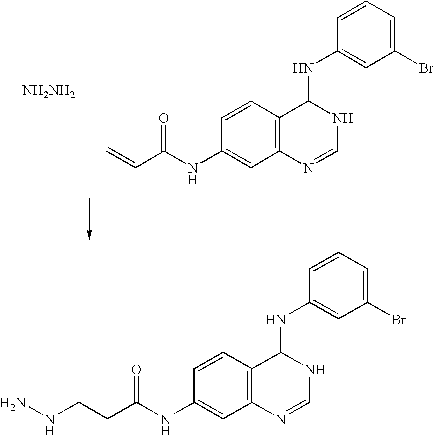

Step 3. Cy7-NHS ester (0.5 mmol), the conjugate compound from step 2 (0.5 mmol) and N-methylmorpholine (70 mg) are dissolved in DMF (30 ml). The mixture is stirred at 40° C. for 3 days. The Cy7 amide conjugate is isolated by flash chromatography (silica, hexane, ethyl acetate, methanol).

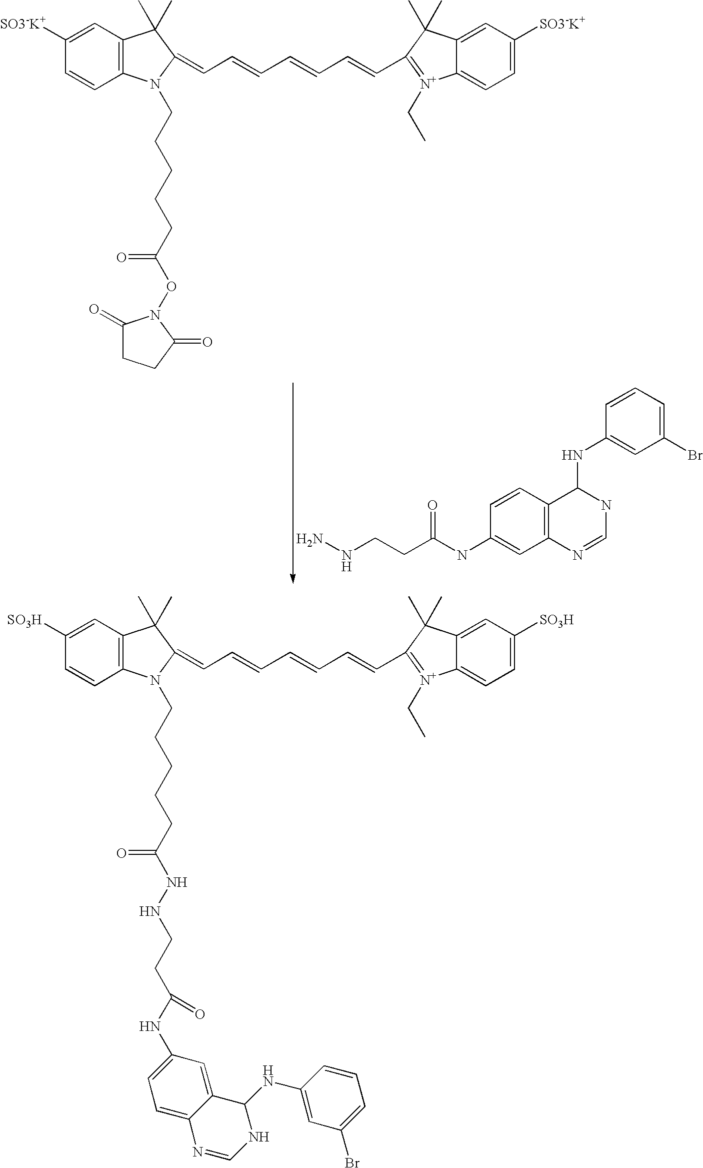

### Example 6

### Inhalation Formulation

The contrast agent from example 5 is filled into a powder inhalation device, e.g. same type of device as the Pulmicort Turboinhaler® from Astra Zeneca. The device contains 200 doses of 0.4 mg of the contrast agent. A contrast dose for diagnosis of lung cancer is typically 0.4 mg to 20 mg.

### Example 7

### Contrast Agent with Affinity for Integrins: RGD Peptide Linked to Cy5.5

Step 1. Assembly of Amino Acids

The peptide sequence Asp-D-Phe-Lys-Arg-Gly was assembled on an Applied Biosystems 433A peptide synthesizer starting with 0.25 mmol Fmoc-Gly-SASRIN resin. An excess of 1 mmol pre-activated amino acids (using HBTU; O-Benzotriazol-1-yl-N,N,N′,N′-tetramethyluronium hexafluorophosohate) was applied in the coupling steps. The cleavage of the fully protected peptide from the resins was carried out by treatment of the resin with three portions of 35 mL of 1% trifluoroacetic acid (TFA) in dichloromethane (DCM) for 5 minutes each. The filtrates containing the peptide was immediately neutralised with 2% piperidine in DCM. The organics were extracted with water (3×100 mL), dried with MgSO₄ and evaporated in vacuo. Diethyl ether was added to the residue and the precipitate washed with ether and air-dried affording 30 mg of crude protected peptide. The product was analysed by analytical HPLC (conditions: Gradient, 20-70% B over 10 min where A=H₂O/0.1% TFA and B=CH₃CN/0.1% TFA; flow, 2 mL/min; column, Phenomenex Luna 3μ 5×4.6 mm; detection, UV 214 nm; product retention time 7.58 min). Further product characterisation was carried out using electrospray mass spectrometry (MH⁺ calculated, 1044.5; MH⁺ found, 1044.4).

Step 2. N—C Cyclisation

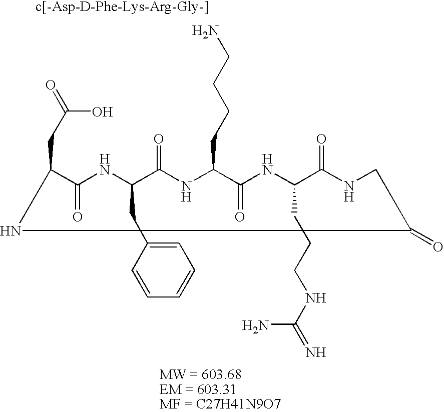

30 mg of the fully protected peptide, 16 mg of PyAOP, 4 mg of HOAt and 6 μL of N-methylmorpholine (NMM) were dissolved in dimethylformamide/DCM (1:1) and stirred over night. The mixture was evaporated in vacuo and diethyl ether added to the residue. The precipitate was washed with ether and air-dried. The crude cyclic fully protected peptide was treated with a solution of 25. mL TFA containing 5% water, 5% triisopropylsilane and 2.5% phenol for two hours. TFA was evaporated in vacuo and diethyl ether added to the residue. The precipitate was washed with ether and air-dried. Purification by preparative RP-HPLC (0-30% B over 40 min, where A=H₂O/0.1% TFA and B=CH₃CN/0.1% TFA, at a flow rate of 10 mL/min on a Phenbmenex Luna 5μ C18 250×21.20 mm column) of the crude material afforded 2.3 mg pure product peptide. The pure product was analysed by analytical HPLC (conditions: Gradient, 0-15% B over 10 min where A=H₂O/0.1% TFA and B=CH₃CN/0.1% TFA; flow, 2 mL/min; column, Phenomenex Luna 3μ 5×4.6 mm; detection, UV 214 nm; product retention time 6.97 min). Further product characterisation was carried out using electrospray mass spectrometry (MH⁺ calculated, 604.3; MH⁺ found, 604.4).

Step 3. Conjugation of Cy5.5 to RGD Peptide

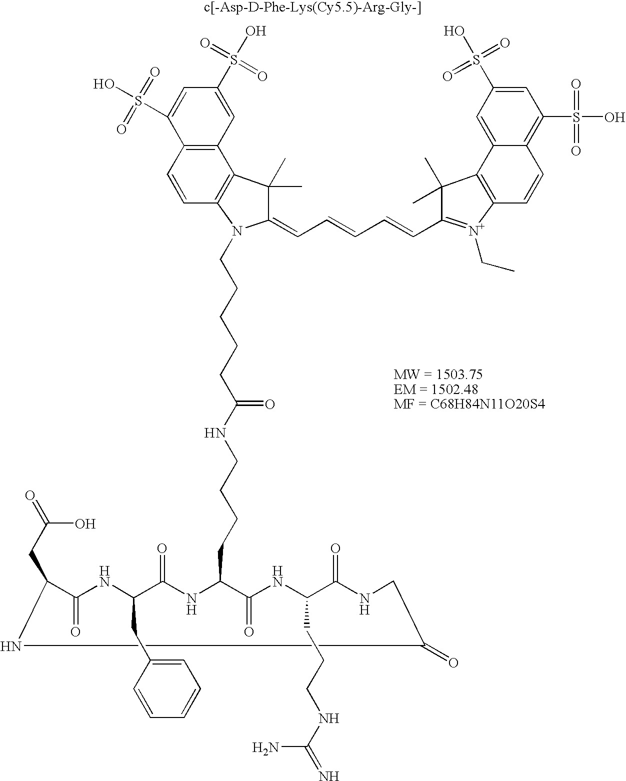

0.6 mg of the RGD peptide, 1.7 mg of Cy5.5 mono NHS ester and 5 μL of NMM were dissolved in 1 mL of dimethylformamide. (DMF) and the reaction mixture stirred for 2 hrs. Diethyl ether was added to the DMF solution and the blue precipitate washed with diethyl ether and air-dried affording 0.7 mg of crude RGD peptide conjugated to Cy5.5.The pure product was analysed by analytical HPLC (conditions: Gradient, 5-50% B over 10 min where A=H₂O/0.1% TFA and B=CH₃CN/0.1% TFA; flow, 0.3 mL/min; column, Phenomenex Luna 3μ 5×2 mm; detection, UV 214 nm; product retention time 8.32 min). Further product characterisation was carried out using electrospray mass spectrometry (MH⁺ calculated, 1502.5; MH⁺ found, 1502.6).

### Example 8

### Synthesis of 3-[(4′-Fluorobiphenyl-4-sulfonyl)-(1-hydroxycarbamoylcyclopentyl)amino]propionic acid (Compound A) Derivatised with Cy5.5-Contrast Agent for Binding to MMP

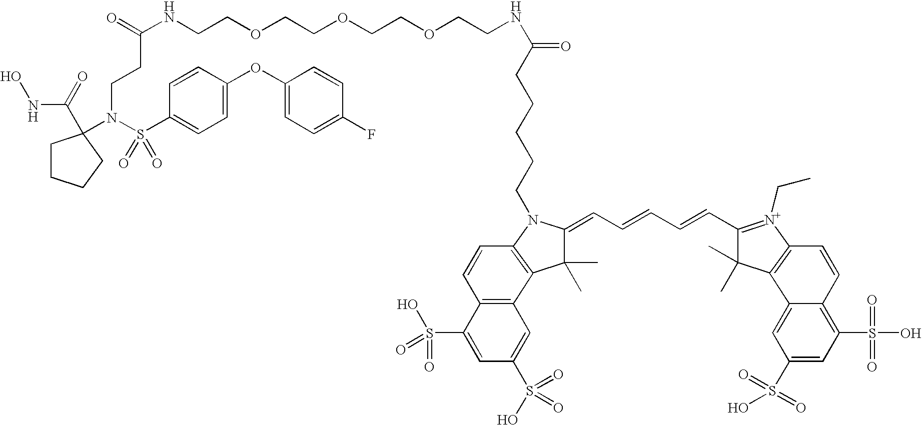

### a) 1.11-Diazido-3.6.9-trioxaundecane

A solution of dry tetraethylene glycol (19.4 9, 0.100 mol) and methanesulphonyl chloride (25.2 g, 0.220 mol) in dry THF (100 ml) was kept under argon and cooled to 0° C. in an ice/water bath. To the flask was added a solution of triethylamine (22.6 g, 0.220 mol) in dry THF (25 ml) dropwise over 45 min. After 1 hr the cooling bath was removed and stirring was continued for 4 hrs. Water (60 ml) was added. To the mixture was added sodium hydrogencarbonate (6 g, to pH 8) and sodium azide (14.3 g, 0.220 mmol), in that order. THF was removed by distillation and the aqueous solution was refluxed for 24 h (two layers formed). The mixture was cooled and ether (100 ml) was added. The aqueous phase was saturated with sodium chloride. The phases were separated and the aqueous phase was extracted with ether (4×50 ml). Combined organic phases were washed with brine (2×50 ml) and dried (MgSO₄). Filtration and concentration gave 22.1 g (91%) of yellow oil. The product was used in the next step without further purification.

### b) 11-Azido-3.6.9-trioxaundecanamine

To a mechanically, vigorously stirred suspension of 1,11-diazido-3,6,9-trioxaundecane (20.8 g, 0.085 mol) in 5% hydrochloric acid (200 ml) was added a solution of triphenylphosphine (19.9 g, 0.073 mol) in ether (150 ml) over 3 hrs at room temperature. The reaction mixture was stirred for additional 24 hrs. The phases were separated and the aqueous phase was extracted with dichloromethane (3×40 ml). The aqueous phase was cooled in an ice/water bath and pH was adjusted to ca 12 by addition of KOH. The product was extracted into dichloromethane (5×50 ml). Combined organic phases were dried (MgSO₄). Filtration and evaporation gave 14.0. g (88%) of yellow oil. Analysis by MALDI-TOF mass spectroscopy (matrix: □-cyano-4-hydroxycinnamic acid) gave a M+H peak at 219 as expected. Further characterisation using ¹H (500 MHz) and ¹³C (125 MHz) NMR spectroscopy verified the structure.

### c) Linking Compound A to PEG(4)-N₃

To a solution of compound A (CP471358, Pfizer, 41 mg, 87 μmol) in DMF (5 ml) were added 11-azido-3,6,9-trioxaundecanamine (19 mg, 87 μmol), HATU (Applied Biosystems, 33 mg, 87 μmol) and DIEA (Fluka, 30 μl, 174 μmol). After one hour reaction time the mixture was concentrated and the residue was purified by preparative HPLC (column Phenomenex Luna C18(2) 5 μm 21.2×250 mm, solvents: A=water/0.1% TFA and B=acetonitrile/0.1% TFA; gradient 30-60% B over 60 min; flow 10.0 ml/min, UV detection at 214 nm), giving 33.9 mg (59%) of product after lyophilisation. LC-MS analysis (column Phenomenex Luna C18(2) 3 μm 50×4.60 mm, solvents: A=water/0.1% TFA and B=acetonitrile/0.1% TFA; gradient 20-100% B over 10 min; flow 1 ml/min, UV detection at 214 nm, ESI-MS) gave a peak at 4.88 min with m/z 667.4 (MH⁺) as expected.

### d) Synthesis of Compound A-PEG(4)-NH₂

To a solution of the PEG(4)-N₃ compound from c) (4.7 mg, 7.0 μmol) in methanol (4 ml) was added Pd/C (Koch-Light, ca 10 mg) added. The mixture was stirred at room temperature under hydrogen atmosphere (1 atm) for 10 min. The mixture was filtered and concentrated. LC-MS analysis (column Phenomenex Luna C18(2) 3 μm 50×4.60 mm, solvents: A=water/0.1% TFA and B=acetonitrile/0.1% TFA; gradient 20-100% B over 10 min; flow 1 mlmin, UV detection at 214 nm, ESI-MS) gave a peak at 4.17 min with m/z 641.4 (MH⁺) as expected. The product was used directly in the next step without further purification.

### e) Conjugation of Cy 5.5

To a solution of the amine from d) (1.0 mg, 1.5 μmol) in DMF (0.2 ml) was added Cy 5.5-NHS (Amersham Biosciences, 1.0 mg, 1.0 μmol) and N-methylmorpholine (1 μl, 9 μmol). The reaction mixture was stirred for 48 h. MS analysis of the solution gave a spectrum showing starting material and the conjugated product at m/z 1539.7 (M⁺⁾, expected 1539.4.

### Example 9

### Cy5-VEGF

Five micrograms of vascular endothelial growth factor (VEGF-121, cat. no. 298-VS/CH) (carrier-free, from R&D Systems) were dissolved in 19 μl of 0.02 M borate buffer, pH 8.5. To this solution was added 2.5 nmol of the N-hydroxysuccinimide ester of a carboxylic acid derivative of Cy5 (Amersham Biosciences), dissolved in 5 μl of the same buffer. The reaction mixture,was incubated for one hour in the dark at room temperature. Unreacted dye was separated from the fluorescent protein derivative by centrifuging through a Micro-Spin 6 gel filtration column (Bio-Rad, exclusion limit about 6 kDa). The eluate fluoresced with excitation light at 646 nm, the emission being measured at 678 nm. The product was a fluorescent targeting molecule for the VEGF receptor.

### Example 10

### Cy5-TIMP-1

Five micrograms of tissue inhibitor of metalloproteinases-1 (TIMP-1, cat. no. 970-TM) (carrier-free, from R&D Systems) were dissolved in 25 μl of 0.02 M borate buffer, pH 8.5. To this solution was added 2.5 nmol of the N-hydroxysuccinimide ester of a carboxylic acid derivative of Cy5 (Amersham Biosciences), dissolved in 5 μl of the same buffer. The reaction mixture was incubated for one hour in the dark at room temperature. Unreacted dye was separated from the fluorescent protein derivative by centrifuging through a Micro-Spin 6 gel filtration column (Bio-Rad, exclusion limit about 6 kDa). The eluate fluoresced with excitation light at 646 nm, the emission being measured at 678 nm. The product was a fluorescent targeting molecule for matrix metalloproteinases.

### Example 11

### Fluorescein-TIMP-1

Five micrograms of tissue inhibitor of metalloproteinases-1 (TIMP-1, cat. no. 970-TM) (carrier-free, from R&D Systems) were dissolved in 25 μl of 0.02 M borate buffer, pH 8.5. To this solution was added 2.5 nmol of the N-hydroxysuccinimide ester of a carboxylic acid derivative of fluorescein (Fluka), dissolved in 5 μl of the same buffer. The reaction mixture was incubated for one hour in the dark at room temperature. Unreacted dye was separated from the fluorescent protein derivative by centrifuging through a Micro-Spin 6 gel filtration column (Bio-Rad, exclusion limit about 6 kDa). The eluate fluoresced with excitation light at 485 nm, the emission being measured at 538 nm. The product was a fluorescent targeting molecule for matrix metalloproteinases.

### Example 12

### Cy5-EGF

Sixty micrograms of epidermal growth factor (EGF, cat. no. 236-EG, 10 nmol) (from R&D Systems) were dissolved in 10 μl of 0.02 M borate buffer, pH 8.5. To this solution was added 10 μl buffer and 50 nmol of the N-hydroxysuccinimide ester of a carboxylic acid derivative of Cy5 (Amersham Biosciences). The reactive dye was dissolved in 5 μl of the same buffer, mixed 1:1 with dioxan. The reaction mixture was incubated for one hour in the dark at room temperature. Unreacted dye was separated from the fluorescent protein derivative by centrifuging through a Micro-Spin 6 gel filtration column (Bio-Rad, exclusion limit about 6 kDa). The eluate, which was bright blue, fluoresced with excitation light at 646 nm, the emission being measured at 678 nm. The product was a fluorescent targeting molecule for the epidermal growth factor receptor.

### Example 13

### Cy7.5-EGF

Sixty micrograms of epidermal growth factor (EG F, cat. no. 236-EG, 10 nmol) (from R&D Systems) were dissolved in 10 μl of 0.02 M borate buffer, pH 8.5. To this solution was added 10 μl buffer and 50 nmol of the N-hydroxysuccinimide ester of a carboxylic acid derivative of Cy7.5 (Amersham Biosciences). The reactive dye was dissolved in 5 μl of the same buffer, mixed 1:1 with dioxan. The reaction mixture was incubated for one hour in the dark at room temperature. Unreacted dye was separated from the fluorescent protein derivative by centrifuging through a Micro-Spin 6 gel filtration column (Bio-Rad, exclusion limit about 6 kDa). The eluate, which was dark green, fluoresced with excitation light at 700 nm, the emission being measured at 790 nm. The product was a fluorescent targeting molecule for the epidermal growth factor receptor.

### Example 14

### Fluorescein-EGF

Sixty micrograms of epidermal growth factor (EGF, cat. no. 236-EG, 10 nmol) (from R&D Systems) were dissolved in 10 μl of 0.02 M borate buffer, pH 8.5. To this solution was added 1.0 μl buffer and 50 nmol of the N-hydroxysuccinimide ester of a carboxylic acid derivative of fluorescein (Fluka), dissolved in 5 μl of dioxan. The reaction mixture was incubated for one hour in the dark at room temperature. Unreacted dye was separated from the fluorescent protein derivative by centrifuging through a Micro-Spin 6 gel filtration column (Bio-Rad, exclusion limit about 6 kDa). The eluate, which was yellow, fluoresced with excitation light at 485 nm, the emission being measured at 538 nm. The product was a fluorescent targeting molecule for the epidermal growth factor receptor.

What is claimed is:

1. An optical imaging contrast agent with affinity for an abnormally expressed biological target associated with lung cancer.

2. A contrast agent as claimed in claim 1 wherein the molecular weight is below 14 000 Daltons.

3. A contrast agent as claimed in claim 1 of formula I
V-L-R, (I)
wherein V is one or more vector moieties having affinity for an abnormally expressed target in lung cancer, L is a linker moiety or a bond and R is one or more reporter moieties detectable in optical imaging.

4. A contrast agent as claimed in claim 1 comprising a contrast agent substrate, wherein the target is an abnormally expressed enzyme, such that the contrast agent changes pharmacodynamic properties and/or pharmacokinetic properties upon a chemical modification from a contrast agent substrate to a contrast agent product upon a specific enzymatic transformation.

5. A contrast agent as claimed in claim 1 having affinity for any of the targets selected from galectin-3, cancer antigen 125 (CA125), cathepsin L, MUC1, caspase-9 and -3, cyclo-oxygenase-2 (COX-2), glutathione-S-transferase (GST), the angiopoietin receptors, integrin αvβ3, vascular endothelial growth factor receptor (VEGF), HER2/epidermal growth factor receptor (EGFR), MDR, urokinase plasminogen activator receptor and cyclin D1.

6. A contrast agent as claimed in claim 3 wherein V is selected from peptides, peptoid moieties, oligonucleotides, oligosaccharides, fat-related compounds and traditional organic drug-like small molecules.

7. A contrast agent as claimed in claim 3 wherein R is a dye that interacts with light in the wavelength region from the ultraviolet to the near-infrared part of the electromagnetic spectrum.

8. A pharmaceutical composition for optical imaging of lung cancer comprising a contrast agent as defined in claim 1 together with at least one pharmaceutically acceptable carrier or excipient.

9. Use of a contrast agent as claimed in claim 1 for the manufacture of a diagnostic agent for use in a method of optical imaging of lung cancer involving administration of said diagnostic agent to an animate subject and generation of an image of at least part of said subject.

10. A method of optical imaging of lung cancer of an animate subject involving administering a contrast agent as defined in claim 1 to the subject and generating an optical image of at least a part of the subject to which said contrast agent has distributed.

11. The method as claimed in claim 10 wherein the optical imaging is for diagnosis of lung cancer, for follow up of the progress of lung cancer development, for follow up of treatment of lung cancer or for surgical guidance.

12. The contrast agent as defined in claim 1 wherein the contrast agent is used for optical imaging of lung cancer.

13. (canceled)
